(19) United States
(12) Patent Application Publication
(10) Pub. No.: US 2007/0122356 A1
(43) Pub. Date: May 31, 2007

(54) GLASS COMPOSITIONS AS AN ANTIMICROBIAL ADDITIVE FOR DENTAL MATERIALS

(75) Inventors: Susanne Kessler, Ergolding (DE); Jorg Hinrich Fechner, Mainz (DE); Karine Senschal, Mainz (DE); Jose Zimmer, Losheim am See (DE)

Correspondence Address: BAKER & DANIELS LLP;111 E. WAYNE STREET, SUITE 800, FORT WAYNE, IN, 46802, US

(73) Assignee: SCHOTT AG, Mainz (DE)

(21) Appl. No.: 10/599,807

(22) PCT Filed: May 25, 2005
(86) PCT No.: PCT/EP05/05632

§ 371 Date: Oct. 10, 2006

(30) Foreign Application Priority Data
May 29, 2004 (DE) 10 2004 026 432.5

(51) Int. Cl. A61K 8/19

(52) U.S. Cl. 424/49; 523/122

(57) ABSTRACT

The invention relates to a use of glass compositions having an antimicrobial and/or disinfectant effect in materials used for restoring teeth, excluding implants.

The invention relates to antimicrobial additives for materials for restoring teeth, for example antimicrobial additives for dental glasses as well as antimicrobial materials for restoring teeth, so-called antimicrobial dental glasses. The materials for restoring teeth comprise in particular materials for filling teeth, wherein the materials for filling teeth comprise e.g. glasionomer cement, composites or compomer. Furthermore, materials for restoring teeth also include additives, in particular antimicrobial additives, in coating or screening materials for ceramic dental structures as well as dental glasses. Dental glasses are for example disclosed in DE 4323143 C1, the content of which was taken into full consideration in this application.

These antimicrobial additives are antimicrobial and/or disinfecting glass compositions or glass ceramic.

The glass compositions are preferably added as powder, fiber, flakes or balls.

These types of antimicrobial additives are used in particular in the area of materials for filling teeth.

In accordance with the Journal de l' Association dentaire canadienne, October 1999, Vol. 65, No. 9, pgs. 500-504, the materials for filling teeth are subdivided into the three classes glasionomer cements, composites and compomers, but are not restricted to these. An expert is familiar with other materials for filling teeth, which can also be used here.

The aforementioned article was taken into full consideration in this application.

In accordance with the Journal de l' Association dentaire canadienne, October 1999, Vol. 65, No. 9, pages 500-504, composites as materials for filling teeth unite two different materials, which together, e.g. as a mixture, develop properties that each material in and of itself does not have. Composites, as known from the state of the art, comprise a resin matrix and different inorganic filler materials.

The resin matrix of a composite is made up of a mixture of different monomers, which result in different properties or property gradations depending on the quantity ratio in connection with the type and mixture of the filler materials.

The resin matrix mainly consists of acrylate monomers PMMA (polymethylmethacrylate), TEGDMA (triethylenglycoldimethacrylate) and BIS-GMA (bisphenol glycidyl methacrylate composite). These types of resin systems are often cured using light. Further components of the resin matrix are often retarders, stabilizers, initiators. Chemically curable systems are also known.

Glasses, (glass) ceramic, quartz, sol-gel materials and aerosols are mainly used as filler materials.

The filler material is embedded in the matrix in order to control the physical and chemical behavior or the compound, i.e. the composite. The filler materials improve in particular the polymerization shrinkage and improve for example the mechanical properties, such as E-module, bending strength, hardness and abrasion resistance.

The curing of the material takes place through chemical reactions, triggered by the mixing of different components, light or heat. Reactive radicals are formed under the influence of light, for example the light of a UV lamp, a halogen lamp, a plasma lamp or an LED lamp (light-emitting diode), in particular an LED, which emits wave lengths in blue, and in connection with additives. These radicals start e.g. a chain reaction, in which the monomers of the matrix material, e.g. Bis-GMA, are combined via a radical intermediate product into longer and longer chain molecules and the plastic is thus cured. Thus, the process concerns “radical polymerization.” In radical polymerization, the intermediate product attaches itself to the carbon double bond of another monomer. This again creates a radical, etc. so that a chain reaction occurs.

Furthermore, it is preferred that the filler materials of the composite are not identifiable, which requires the best possible modification of the calculation indices of the cured resin and the filler material. The smallest possible particle size of the filler material is also preferred, which in turn improves the ability to polish the entire filling, i.e. the composite. Suitable are particles with particle sizes smaller than 100 μm, preferably smaller than 50 μm, even more preferably smaller than 10 μm. If the particle size is less than a value of 2 nm, preferably less than 5 nm, even more preferably less than 10 nm, then the mechanical properties of the composites are too weak.

For the filler materials, it is also possible to use mixtures of particles of different size, for example a powder with a medium particle size in the nm range and a powder with a medium particle size in the pm range. With this type of mixture, the ability to polish the composite and the mechanical properties of the composite are increased.

The composites in accordance with the state of the art have low polymerization shrinkage. If the polymerization shrinkage is too high, high tension would occur between the wall of the tooth and the filling. If the polymerization shrinkage is too large, the wall of a tooth can even break in extreme cases. If the adhesion between the filling and the wall of the tooth is poor and/or if the material for the filling of the tooth shrinks too much, then it can lead to the formation of edge gaps, which in turn lead to secondary caries. Materials current available on the market shrink by approx. 1.5-2%.

In particular for applications in the front tooth area, the composites have a color and translucence so that the composite cannot be differentiated from the surrounding healthy tooth substance. Thus, the material is primarily adjusted to match the color of the healthy tooth substance and the translucence primarily matches that of a natural tooth.

Regarding the mechanical properties, it is advantageous if the fracture-mechanical properties are such that the filling is not worn too much during chewing and that the opposite lying tooth is not damaged.

Regarding the thermal expansion of the composite, it is advantageous if this is primarily adjusted for the thermal expansion of the tooth substance.

Regarding the chemical resistance of the composite, it is designed such that the composite has sufficient stability against basic attacks.

Furthermore, the composite has an X-ray opacity so that the filling can be differentiated from healthy tooth material and any secondary caries in an X-ray image.

Regarding the rheology, the resin is advantageously thixotrop, i.e. viscosity decreases as pressure increases, and then increases again. This behavior is advantageous since the resin must be filled into the cavity from cartridges but must also be as inherently stable as possible before hardening.

The term glasionomer cement is defined in ISO 7484, the content of which is taken into full consideration in this application.

Aqueous poly-(carbonic acid)-cement compositions are known e.g. as glasionomer cement and are already used in dentistry. Glasionomer cements comprise a polymer, which contains free carbonic-acid groups, typically a homo- or co-polymer of an acrylic acid, and an ion-releasing glass, such as a calcium aluminum fluorosilicate glass.

Glasionomer cements are formed via a acid-base reaction in an aqueous solution. In the presence of water, the glass releases polyvalent metal ions, such as aluminum and calcium ions. These serve to link the polymer. A stiff, gelatin-like structure is obtained in this manner. At the same time, the material in the glass reacts with water and forms silicic acid. A cement suitable for dental applications is formed as a result of this gel-forming reaction.

Since glasionomer cements are brittle and not very elastic, their use is extremely limited based on the insufficient mechanical properties. In order to improve the mechanical properties of glasionomer cements, it is e.g. known to modify the matrix. For this, either unsaturated carbon-carbon bonds were engrafted on a polyalkenoate main structure or (di) methacrylate monomer(s) were included in the composition or both were performed. Unsaturated carbon-carbon bonds enable a covalent linking of the matrix via radical polymerization (chemically or via light rays). A covalently linked matrix clearly improves the mechanical properties of the attached cement. The dental pulp tolerates this cement well. However, problems occurred with respect to the biocompatibility, since undesired resin components can be released, such as hydroxyethyl methacrylate or HEMA. These compounds are known as resin-modified glass ionomer cements or RMGICs, although their structure would be better described as resin-modified glass-polyalkenoate cements. These RMGICs are based on water, an acid-base reaction is the main setting mechanism and they thereby retain their ability to bond to hard tooth material via the carboxyl groups of the polyalkenoate components. Their fluoride release is similar to the GICs.

Furthermore, polymerizable cements are know as they are e.g. described in EP-A-0219058 and are know under the name “compomer” and “plastic-reinforced glasionomer cement”.

The plastic-reinforced glasionomer cement compomer is a material that combines the advantages of a composite material (the syllable “comp” in the name) with that of a glasionomer (the syllable “omer” in the name). The material comprises dimethylmethacrylate monomers with two carboxyl groups and a filler material, which is primarily an ion-releasing glass. The ratio of carboxyl groups to the carbon atoms of the backbone is 1:8. The composition is water-free and the ion-releasing glass is partially silanized in order to ensure a bonding with the matrix. These materials labeled as compomers are set via a radical polymerization, but cannot bond to hard tooth material and have a much lower fluoride release than glasionomer cements.

They have a lower elastic bending module, a low bending strength, pressure resistance and tensile strength and low hardness. The compomers can be used as adhesives in orthodontics, as an amalgam-bonding system and in the area of veterinary medicine. Since these materials cannot set via an acid-base reaction and can also not bond to hard tooth material, they should actually not be classified as glasionomer cements, since they represent a completely different material.

Furthermore, components are often not entirely correctly labeled as “hybrid glasionomers,” “light-cured GICs,” or “resin-modified glasionomers”, i.e. the actual “resin-modified glasionomers.” The term “poly-acid-modified composite resin” is also used.

All types of materials for filling teeth, especially such as glasionomers, composites and compomers, can contain aerosols, e.g. pyrogenic silicic acid that are used to establish the rheology as filler materials or additives in addition to the inert or reactive dental glasses as further filler material. In contrast to the ground glass powder, the aerosols are spherical in form and have particle sized of approx. 50-300 nm.

Pigments for setting the tooth color and materials for achieving X-ray opacity can be included as further filler materials. Examples of these types of materials are BaSO₄, ZrO₂, YbF₃.

Sol-gel materials, such as Zr silicates, which have X-ray opacity can also be used as filler materials.

Furthermore, organic fluorescence pigments for restoring the fluorescence properties of the natural tooth can also be provided.

It was disadvantageous for the known materials in the field of dentistry, in particular the glasionomer cements, the composites and the compomers that they have no antimicrobial effect and thus do not provide enough protection from antimicrobial triggered dental diseases such as secondary caries, root infections or periodontosis.

The antimicrobial, anti-inflammatory and wound-healing effect of glasses, in particular glass powder made if it became known from the following documents, the content of which is taken into full consideration in this application:
WO 03/018496
WO 03/018498
WO 03/018499

The W003/018496 and the W003/018499 show an anti-inflammatory and wound-healing silicate glass powder.

Antimicrobial, anti-inflammatory glass and glass powder, the glass composition of which contains more than 10 ppm of iodine, became known from W003/018498. The use of alkali-earth alkali glasses without Ag, Zn, Cu in dental materials is known from W002/072038 and EP-A-1365727, the content of which was taken into full consideration in this application.

The object of the invention is to overcome the disadvantages of the state of the art and in particular to provide additives for dental materials that have an antimicrobial and disinfecting, anti-inflammatory and wound-healing effect.

This object is solved in accordance with the independent claims. Advantageous embodiments are the subject of the dependent claims.

In a particularly preferred embodiment, the antimicrobial additives, which are also called antimicrobial dental glass powder below, function as glasionomers, i.e. in addition to the antimicrobial effect, they also function as initiators for a polymerization of monomers, i.e. make available the ions necessary for the curing reaction to a glasionomer cement, e.g. the Ca²⁺, Al³⁺ions. For example, the lixiviation of Ca²⁺, Al³⁺ions together with e.g. the polycarboxylic acids of the plastics of the cements causes the curing.

In an alternative embodiment, the antimicrobial glass itself has no ionomer properties, but rather functions as an additive material, which makes available the antimicrobial effect. It is an inert antimicrobial dental glass powder, as is e.g. used in composites. If the antimicrobial dental glass powder is only used as an additive material, i.e. as an inert antimicrobial dental glass powder, then the polymerization of the monomers can be achieved e.g. through light e.g. UV irradiation or heat.

In another embodiment, the inert or even the reactive antimicrobial dental glass powder is designed such that the shrinkage of the glasionomer cement, composite or compomer resulting from the polymerization decreases or X-ray opacity is achieved. It is even possible to design the antimicrobial dental glass such that a remineralization of the dental enamel is supported.

Of course, mixtures of antimicrobial dental glass powder in accordance with the invention with other dental fillers, e.g. conventional dental glasses, are also possible.

In a preferred embodiment of the invention, the thermal expansion coefficient, the CTE of the antimicrobial dental glass powder is very small and lies between 3·10⁻⁶/K and 8·10⁻⁶/K.

The breaking index of the antimicrobial dental glass powder is preferably selected such that the breaking index is primarily adjusted for that of the matrix, whereby the glass powder itself is primarily free of coloring ions.

In a further embodiment, the glass powder surface of the antimicrobial dental glass powder is silanized so that a chemical bond between the filler material particles and the resin matrix is enabled. This in turn results in improved mechanical and rheological properties of the filling or the formulation.

It is especially preferred if the antimicrobial dental glass powder has a good, chemical and hydrolytic resistance as well as a high X-ray opacity (XO).

A high X-ray opacity is achieved in particular through the addition of heavy elements, such as Sr or Ba.

In order to improve the aesthetics and the polishability, small particle sizes of the antimicrobial dental glass powder of d50 between 0.4-5 μm are preferred.

Especially preferred are embodiments that have long-term antimicrobial effects.

It is especially preferred is the materials have a high antimicrobial and disinfecting effect, but do not release any or only very few quantities of antimicrobial ions, such as zinc or silver.

The antimicrobial glass in accordance with the invention is preferably used in coating, filling or screening materials for dentistry.

In contract to implant materials, which are inserted into the jaw, the materials described in this application are preferably used in or on the tooth.

In a special application in glasionomer cements, the cements comprise the antimicrobial glass additive or the antimicrobial glass ceramic in a concentration in the range of 0.01-99.5 percentage of weight. 0.1 to 80 wt. % are preferred, and 1 to 21 wt. % of the antimicrobial glass additive or glass ceramic additive are especially preferred in glasionomer cements.

The antimicrobial glasses in accordance with the invention can also be missed with known glass powders, which are used in dental filling materials.

The particle size of the antimicrobial glass powder at d50 values is e.g. larger than 0.1 μm, preferably larger than 0.5 μm, even more preferably larger than 1 μm.

The particle size of the antimicrobial glass powder at d50 values is e.g. smaller than 200 μm, preferably smaller than 100 μm, even more preferably smaller than 20 μm. The most preferred are particle size distributions with particle sizes larger than 0.1 μm and smaller than or equal to 10 μm, in particular due to the better polishability between 0.1-1.5 μm.

The glasses contain in preferred embodiment examples antimicrobial elements or ions, such as Ag, Zn, Cu. The release rates of the antimicrobial ions are so low in the glass matrices that no health risk exists; however, on the other hand, a sufficient antimicrobial effect is achieved.

For example, during the release of silver as an antimicrobial ion, sufficient release is achieved for an antimicrobial effect, which does not yet lead to damaging health effects, if the release rates of e.g. silver in water from the glasses in accordance with the invention lie below 1000 mg/l, preferably <500 mg/l and even more preferably <20 mg/l. In a particularly preferred embodiment, it is <10 mg/l.

If the antimicrobial glass is inserted into a composite material in accordance with the invention, then even smaller quantities are released in contact with fluid such as water or saliva than from the free glass in water. Release rates of e.g. silver in water from the composite in accordance with the invention or glasionomer cement or compomer lie e.g. below 10 mg/l, preferably <1 mg/l, even more preferably <0.1 mg/l.

In order to make available a sufficient antimicrobial effect, the release rates lie e.g. above 0.0001 mg/l, preferably above 0.001 mg/l and even more preferably above 0.01 mg/l. Basis glasses are phosphate, borate and silicate glasses that do no have too high of a chemical resistance.

It is advantageous that the refractive index of these glasses can be adjusted.

In order to obtain an antimicrobial and disinfecting effect, the concentration of ions, such as Ag, Zn, Cu, in the glasionomer is larger than 0.01 wt. %, preferably larger than 0.1 wt. %, even more preferably larger than 0.5 wt. %. In contrast to WO 93/17653A1, less than 30 atom % Zn are preferably contained in the glass composition.

If a preferred embodiment consists of a mixture in accordance with the invention made of an antimicrobial glass powder, which is also called an antimicrobial dental glass powder in this application, and a glasionomer and/or a dental glass filler, then the ratio of antimicrobial glass powder/glasionomer and/or dental glass filler >0.0001m, preferably greater than 0.001 and even more preferably greater than 0.01.

If the concentration of antimicrobial glass powder is too low, i.e. if the ratio of antimicrobial glass powder/glasionomer and/or dental glass filler <0.0001, then a sufficient antimicrobial and disinfecting effect of the mixture is no longer achieved.

A ratio of antimicrobial/glasionomer and/or dental glass filler <200 is preferred, <100 is more preferred and <10 is especially preferred.

If the mixture has a ratio of antimicrobial glass powder/glasionomer and/or dental glass filler that is greater than 200, then as a rule sufficient initiation of the polymerization of the monomers by the glasionomer is no longer achieved.

In a special embodiment, the antimicrobial powder, when it comes in contact with water or saliva etc., sets a basic pH, i.e. a pH value >7, through ion exchange with the glass matrix. This neutralizes acids, which are formed through caries bacteria and can attack the tooth or tooth enamel. In particular, this reaction prevents the attack in the spaces between the dental material and the tooth.

The combination of antimicrobial glass powder with especially re-mineralizing glass powders, like a glass powder as disclosed in EP-A-1365727, is possible and preferred. For one, a tight connection between the tooth and the dental material is thereby achieved, and, on the other hand, since re-mineralizing glass powder, like the glass powder from EP-A-1365727, also has a low antimicrobial effect, an antimicrobial synergistic effect is achieved. The use of bioactive glass for the production of a substance for a permanent filling of the tooth is described in EP-A-1365727. The bioactive glass is preferably contained in a bonding, which acts as an adhesive agent between the tooth substance and the filling material, in a glasionomer cement, in a glass/plastic composite, in a composite-reinforced glasionomer cement and/or in a substance for treating the root of the tooth, the neck of the tooth and/or the crown of the tooth and preferably contains fluoride ions.

An antimicrobial effect, for example through the release of Ag, Zn or Cu ions, is not described in the glasionomer cement, in the glass plastic composite, in the composite-reinforced glasionomer cement and/or in the material for treating the root of the tooth, the neck of the tooth and/or the crown of the tooth, which contains the bioactive glass described in EP-A-1365727. It is especially preferred if the glass has a high X-ray opacity.

In a preferred embodiment, the antimicrobial glass additive releases fluoride, such as the glass composition disclosed in WO 03/18499. The selection of this type of antimicrobial glass powder prevents the formation of caries. The antimicrobial glass powder preferably has re-mineralizing properties.

In a further embodiment, the antimicrobial additive itself functions as a glasionomer, i.e. it makes available the ions necessary for the curing reaction into a glasionomer cement, such as the Ca²⁺, Al³⁺ions. The lixiviation of Ca²⁺, Al³⁺ions together with the polycarboxylic acids in plastic cause the cement to harden or cure. For the re-mineralizing properties, glass compositions are preferably used that contain and release Ca and/or phosphor ions and/or sodium and/or bonds containing Ca or phosphor and thus support the re-mineralization of the tooth.

Known glasionomer cements are often made up of a powder/liquid system.

The glasionomer cement is created through a setting reaction of the liquid components with the glasionomers as described below.

As a rule, the organic components are processed into a liquid, which results in the liquid components, which are mixed with the solid components, in particular the powder, in particular the glass powder, the so-called glasionomers, right before use by the dentist. The liquids are made up e.g. of polyacrylic acids, tartaric acid, distilled water, three-resin complexes, such as 2-hydroxyethyl methacrylate (HEMA). Paste/paste systems, in which the components that do not achieve a reaction with the glasionomers or the mixture in accordance with the invention of glasionomers and antimicrobial glass powder, are mixed with it into a paste, e.g. 2-hydroxyethyl methacrylate, dimethacrylate or pigments, are also common. The other components, such as polyacrylic acids, water, pyrogenic silicic acid are mixed in a second paste. The dentist then triggers the setting reaction by intensively mixing the pastes, thereby producing the glasionomer cement.

Reinforced systems are also known, in which e.g. methacrylate-modified polycarboxylic acids are used.

If the cement needs to be dual-hardening, the use of photo-initiators, such as Campherchinon, is possible.

The advantage of a mixture of antimicrobial glass powders with non-anti-microbial glasionomers in accordance with the invention is that the antimicrobial effect of the mixture exceeds the individual antimicrobial effect of the glass powder, since the release of antimicrobial ions, such as AG, from the antimicrobial glass powder is triggered by the ions released from the glasionomer.

Another advantage is that the radical polymerization (initiated by e.g. light or heat), i.e. the polymerization degree and thus the level of stability (e.g. E-module etc.) as well as the kinetics of the polymerization of the cement are synergistically supported by the addition of ion-releasing, antimicrobial powder.

If the composites contain the aforementioned filler materials, the biocide ions such as Ag⁺, Zn²⁺, Cu²⁺, then the entire composite can have an antimicrobial effect through the release of these ions from the glass. Due to the fact that the entire composite has an antimicrobial effect, the formation of secondary caries is prevented, or at least slowed down significantly.

The glass fillers used as the filler material cannot have an antimicrobial effect in and of themselves, but can be part of the mixture of the glass filler and the antimicrobial glass.

In the case of the glasionomer cements, it is also possible that carboxyl-containing groups of the polyalkenoate chains chelate the calcium of the hydroxylapatite layer of the antimicrobial glass powder through the addition of antimicrobial glasses, in order to the set the adhesive into a mineralized hard tooth material. Through the addition of antimicrobial glass powder into a glasionomer cement, it is also possible that a fixed bonding to the tooth enamel substance is created.

Moreover, the ions of the reaction, which are used to set the glasionomer cement, cause calcium, aluminum, sodium, fluoride and silicic-acid ions to be released from acid-soluble glass.

From a structural point of view, a glasionomer cement is a composite, in which the un-reacted glass particles are material fillers and the calcium/aluminum diagonally connected polyalkenoate chains form the matrix. The glass particles surrounded by the matrix then represent a bond between the filler and the matrix.

The ionic bonds are responsible for the linking of the polymer chains and the setting of the glasionomer cement. The large number of secondary bonds plays an important role in the setting of the mechanical properties of the cement.

Glasionomer cements are brittle and have a low elasticity mode; they are weak under tension and have a low tensile strength. Due to their poor mechanical properties, their use as tooth restoration material is limited.

One possibility for improving the mechanical properties of glasionomer cements is an improved matrix. Advancements were made with respect to the state of the art, in that antimicrobial glasses were used to strengthen the matrix, which resulted in a solid bond with the hard tooth material.

In the case of compomers, the addition of antimicrobial glass powder causes a reduction in shrinkage. Furthermore, the mechanical properties of glasionomers are improved and the composites achieve a strong bonding effect.

The invention is explained below using exemplary embodiments without being restricted to them.

Borosilicate glasses are suitable as antimicrobial glass additives for a glasionomer in a glasionomer cement, in particular in the form of an antimicrobial glass powder. Exemplary embodiments for borosilicate base glasses that were not subjected to any special treatment for achieving a phase-mixed system must be specified first.

The glasses were obtained in that a glass was melted from the raw materials and was then shaped into ribbons. These ribbons were further processed into powder with a particle size of d50=4 μm by means of dry grinding.

Table 1 shows glass compositions in wt. % based on the oxide of borosilicate glass in accordance with the invention, which can be ground into a glass powder and used in the glasionomer cement.

TABLE 1
Compositions in wt. % based on the oxide of borosilicate glass in accordance with the invention

| | A1 | A2 | A3 | A4 | A5 | A6 | A7 | A8 | A9 | A10 | A11 | A12 | A13 | A14 | A15 | A16 | A17 | A18 |
|---|---|---|---|---|---|---|---|---|---|---|---|---|---|---|---|---|---|---|
| SiO₂ | 63.5 | 63.5 | 62.5 | 71 | 61 | 69 | 61 | 61 | 64.5 | 60.99 | 56.2 | 63.5 | 77 | 70 | 57 | 63.5 | 61 | 65 |
| B₂O₃ | 30 | 29.9 | 28 | 21 | 21 | 16 | 22 | 36 | 25.5 | 22 | 18 | 29 | 14.5 | 10.7 | 27 | 29 | 37 | 33 |
| Al₂O₃ | | | 4 | | | 2.75 | | | | | 6.63 | | 4 | 4 | | | | |
| P₂O₅ | | | | | | 2.75 | | | | | | | | | | | | |
| Na₂O | 6.5 | 6.5 | | 7 | | 6 | 3 | 2.99 | 4.7 | 5 | 3.7 | 6.5 | 3.5 | 2.8 | 6 | 5.5 | | |
| Li₂O | | | | | | | | | | | 1.84 | | | | | | | |
| K₂O | | | | | | | 4 | | | | 5.64 | | 1 | 3.6 | | | | |
| BaO | | | 5 | | | | | | | | | | | | | | | |
| CaO | | | | | | 3 | | | | | | | | 2.1 | | | | |
| MgO | | | | | | | | | | | | | | | | | | |
| SrO | | | | | | | | | | | | | | | | | | |
| ZnO | | | | | 18 | | 9.95 | | | | 0.28 | | | 2.5 | 10 | | | |
| SO₃ | | | | | | | | | | | 5.37 | | | | | | | |
| Ag₂O | | 0.1 | 0.5 | 1 | | 0.5 | 0.05 | 0.01 | 5 | 0.01 | 0.21 | 1 | | | | 2 | 2 | 2 |
| CuO | | | | | | | | | | 2 | 2.07 | | | | | | | |
| GeO₂ | | | | | | | | | | | | | | | | | | |
| TeO₂ | | | | | | | | | | 1 | 0.04 | | | | | | | |
| Cr₂O₃ | | | | | | | | | | 1 | 0.01 | | | | | | | |
| ZrO₂ | | | | | | | | | | | | | | 4.3 | | | | |
| Jod | | | | | | | | | | | 0.01 | | | | | | | |
| Br | | | | | | | | | | | | | | | | | | |
| Cl | | | | | | | | | | | | | | | | | | |
| La₂O₃ | | | | | | | | | 0.3 | | | | | | | | | |

Table 2 shows borosilicate glass that was subjected to a defined temperature process. A defined decomposition into multi-phase systems, in particular a 2-phase system, was achieved through this tempering. The glass was melted from the raw materials as specified for the respective exemplary embodiments in Table 1 and then shaped into ribbons. The tempering specified in Table 2 was then performed at the specified temperatures for the specified time. Table 2 specifies the tempering temperature, the tempering time and the size the decomposed areas, the so-called decomposition size in a 2-phase system, for the different glass compositions as per Table 1.

TABLE 2
Size of the decomposed areas for different glass compositions for different
temperatures and tempering times

| | Glass Composition | Tempering | Temperature | | Decomposition |
|---|---|---|---|---|---|
| Sample | as per Table 1 | to | (° C.) | Time (h) | Size |
| Version 1-a | Version 1 | Ribbon | 560 | 10 | 30 nm |
| Version 1-b | Version 1 | Ribbon | 560 | 20 | 60 nm |
| Version 1-c | Version 1 | Ribbon | 620 | 10 | 40 nm |
| Version 1-d | Version 1 | Ribbon | 620 | 20 | 80 nm |
| Version 2-a | Version 2 | Ribbon | 560 | 10 | 40 nm |
| Version 2-b | Version 2 | Ribbon | 560 | 20 | 100 nm |
| Version 2-c | Version 2 | Ribbon | 620 | 10 | 70 nm |
| Version 2-d | Version 2 | Ribbon | 620 | 20 | 150 nm |
| Version 12a | Version 12 | Ribbon | 560 | 10 | 50 nm |
| Version 12b | Version 12 | Ribbon | 560 | 20 | 150 nm |
| Version 12c | Version 12 | Ribbon | 620 | 10 | 80 nm |
| Version 12d | Version 12 | Ribbon | 620 | 20 | 200 nm |
| Version 14a | Version 14 | Ribbon | 820 | 5 | 40 nm |

The systems in accordance with Table 2 are two-phase systems, whereby the compositions of the two phases are different. The one phase is a phase in which boron is enriched, the other phase is a phase, in which silicon is enriched. The antimicrobial effectiveness increases due to the lower chemical resistance of the boron-rich phase, since the release of antimicrobial ions, such as silver, can take place faster.

ables 3 through 5 specify the antimicrobial effect for different exemplary embodiments of glass compositions as per Table 1. The determination of the antimicrobial effect concerns measurements from the glasses of the glass powders containing the respective glass compositions that were obtained from the ribbon through grinding. A tempering on the ribbon was only used for the glass powder specified in Table 3.

TABLE 3
Antibacterial effect of a glass powder as per Europ. Pharmakopoe
(3^{rd} edition) for a glass composition in accordance with
exemplary embodiment 2 in Table 1 with a particle size of 4 μm
in an aqueous suspension at a concentration of 0.01 wt. %.
The glass was tempered before grinding.

| | E. coli | P. aeruginosa | S. aureus | C. albicans | A. niger |
|---|---|---|---|---|---|
| Start | 350000 | 250000 | 270000 | 333000 | 240000 |
| 2 Days | 0 | 0 | <100 | 0 | 240000 |
| 7 Days | 0 | 0 | 0 | 0 | 180000 |
| 14 Days | 0 | 0 | 0 | 0 | 50000 |
| 21 Days | 0 | 0 | 0 | 0 | 16000 |
| 28 Days | 0 | 0 | 0 | 0 | 4000 |

TABLE 4
Antibacterial effect of a glass powder as per Europ. Pharmakopoe
(3^{rd} edition) for a glass composition in accordance with
exemplary embodiment 12 with a particle size of 4 μm in an
aqueous suspension at a concentration of 0.001 wt. %.
The glass was tempered at 620° C. for 10 hours on the ribbon
before grinding as in exemplary embodiment 12c in Table 2,
so that a glass decomposed in two phases was obtained with
a decomposition size of 80 nm.

| | E. coli | P. aeruginosa | S. aureus | C. albicans | A. niger |
|---|---|---|---|---|---|
| Start | 270000 | 260000 | 260000 | 240000 | 240000 |
| 2 Days | 0 | 0 | 0 | <100 | 180000 |
| 7 Days | 0 | 0 | 0 | 0 | 100000 |
| 14 Days | 0 | 0 | 0 | 0 | 60000 |
| 21 Days | 0 | 0 | 0 | 0 | 12000 |
| 28 Days | 0 | 0 | 0 | 0 | 6000 |

TABLE 5
Antibacterial effect of a glass powder as per Europ. Pharmakopoe
(3^{rd} edition) for a glass composition in accordance with
exemplary embodiment 11 in Table 1 with a particle size of 4 μm
in a aqueous suspension at a concentration of 0.01 wt. %.
The glass was not tempered before grinding.

| | E. coli | P. aeruginosa | S. aureus | C. albicans | A. niger |
|---|---|---|---|---|---|
| Start | 290000 | 220000 | 250000 | 270000 | 280000 |
| 2 Days | 0 | 0 | 100 | <100 | 100000 |
| 7 Days | 0 | 0 | 0 | 0 | 30000 |
| 14 Days | 0 | 0 | 0 | 0 | 22000 |
| 21 Days | 0 | 0 | 0 | 0 | 14000 |
| 28 Days | 0 | 0 | 0 | 0 | 14000 |

In the above tables 3 through 5, the start value describes the number of bacteria used at the beginning of the measurements. If the value is 0, then no bacteria are measurable. This is proof of the antimicrobial effect of the glass powder.

As proof of the release of antimicrobial ions over time, Table 6 specifies the release of Ag ions from glass powder into an aqueous solution.

Table 6 specifies the ion release for Si, Na, B and Ag in mg/L under continuous lixiviation after 1 hour, after 24 hours, after 72 hours and after 168 hours in accordance with exemplary embodiment 2 in Table 1 and 2-c in Table 2 with a particle size of 5 μm, in an aqueous suspension at a concentration of 1 wt. %.

TABLE 6

| | SiO₂ | Na₂O | B₂O₃ | Ag |
|---|---|---|---|---|
| after 1 hour (mg/L) | | | | |
| Version 2 | 227 | 1283 | 6929 | 0.63 |
| Version 2-c | 781 | 3384 | 14019 | 6.1 |
| after 24 hours (mg/L) | | | | |
| Version 2 | 121 | 74 | 274 | 0.035 |
| Version 2-c | 164 | 37.6 | 36.1 | 0.44 |
| after 72 hours (mg/L) | | | | |
| Version 2 | 70.8 | 23.8 | 60.8 | 0.02 |
| Version 2-c | 61.3 | 4.6 | 4.70 | 0.36 |
| after 168 hours (mg/L) | | | | |
| Version 2 | 51.4 | 9.5 | 14.1 | 0.01 |
| Version 2-c | 16.3 | 2.62 | 2.89 | 0.3 |

In this application, continuous lixiviation is understood to mean that after e.g. 72 hours of water flow, in a glass in accordance with an exemplary embodiment 2c, e.g. 0.36 mg/l of silver are still released, as specified in Table 6.

It can be seen that the decomposed glass releases considerably more boron, sodium and, in particular, silver ions than the non-decomposed glass at the beginning of the lixiviation. The antimicrobial effectiveness is increased due to the lower chemical resistance of the boron-containing phase.

The boron-containing phase is the highly reactive phase of the two-phase system with a very fast silver-ion release or a very strong short-term antimicrobial effect. The silicate-containing phase ensures a slow silver release through its higher chemical resistance and the antimicrobial long-term effect of the glass.

As an alternative glass composition, zinc phosphate glass can be used as antimicrobial additives to dental materials. These glass compositions are specified in Tables 8 and 9:

TABLE 8
Compositions (synthesis values) [wt. %] of glass compositions in accordance with the invention

| | A19 | A20 | A21 | A22 | A23 | A24 | A25 | A26 | A27 | A28 | A29 | A30 | A31 | A32 | A33 | A34 | A35 | A36 |
|---|---|---|---|---|---|---|---|---|---|---|---|---|---|---|---|---|---|---|
| P₂0₅ | 66.1 | 70 | 68 | 66.1 | 67 | 75 | 67.5 | 65.9 | 65.9 | 75 | 67 | 72 | 67 | 80 | 65.9 | 66.3 | 66 | 69 |
| SO₃ | | | | | | | | | | | | | | | | | | |
| B₂O₃ | | | | | | | | | | 1 | | | | | | 7.2 | 7 | |
| Al₂O₃ | 6.9 | 7 | 6.5 | 6.9 | 7 | 7 | 7 | 6.2 | 6.2 | 0 | 0 | 5 | 5 | 3 | 6.2 | 0.4 | | 6 |
| SiO₂ | | | | | | | | | | | | | | | | 0.7 | 0.5 | 4 |
| Li₂O | | | | | | | | | | | | | | | | | | |
| Na₂O | 10 | 10.5 | 9 | 10 | 12.2 | 9.0 | 11 | | | | | | | 2.7 | | | | |
| K₂O | | | | | | | | | | | | | | | | | | |
| CaO | | | 8 | | 13 | | | 11.9 | 11.9 | | 11 | 20 | 8 | 5 | | 9.7 | 10 | 3 |
| MgO | | | | | | | | | | 8.5 | | | | | | 13.7 | 13.5 | 15 |
| SrO | | | | | | | | | | | | | | | | | | |
| BaO | | | | | | | | | | 13 | | | | | 11.90 | | | |
| ZnO | 16 | 12 | 8.5 | 10 | | 10 | 13.5 | 15 | 16 | 2 | 22 | 2 | 20 | 9 | 15 | | | |
| Ag₂O | 0.01 | 0.5 | | 0.5 | 0.8 | 2.0 | 1 | 1 | | 0.5 | | 1 | | | 1 | 2 | 2 | 2 |
| CuO | | | | 0.01 | | | | | | | | | | | | | | |
| La₂O₃ | | | | | | | | | | | | | | 0.3 | | | | |
| ZrO₂ | | | | | | | | | | | | | | | | | 1 | 1 |

Table 9 specifies the antimicrobial effect for exemplary embodiment 20 in accordance with Table 8.

TABLE 9
Antibacterial effect of the powder as per Europ. Pharmakopoe
(3^{rd} edition) in 0.001 wt. % aqueous solution.
Exemplary embodiment 25 particle size 4 μm:

| | E. coli | P. aeruginosa | S. aureus | C. albicans | A. niger |
|---|---|---|---|---|---|
| Start | 260000 | 350000 | 280000 | 360000 | 280000 |
| 2 Days | 0 | 0 | 0 | 0 | 0 |
| 7 Days | 0 | 0 | 0 | 0 | 0 |
| 14 Days | 0 | 0 | 0 | 0 | 0 |
| 21 Days | 0 | 0 | 0 | 0 | 0 |
| 28 Days | 0 | 0 | 0 | 0 | 0 |

The exemplary embodiment 25 has a pH value of approx. 5.0 in a 1% aqueous solution.

Table 10 shows the antimicrobial effect for exemplary embodiment 26 in accordance with Table 8. 0.001 wt. % glass powder with a particle size of d50=4 μm of the exemplary embodiment 26 was measured in an aqueous solution.

TABLE 10
Antibacterial effect of the powder as per Europ. Pharmakopoe
(3^{rd} edition) in 0.001 wt. % aqueous suspension:
Exemplary embodiment 26 as per Table 8; particle size 4 μm

| | E. coli | P. aeruginosa | S. aureus | C. albicans | A. niger |
|---|---|---|---|---|---|
| Start | 240000 | 340000 | 240000 | 330000 | 280000 |
| 2 Days | 0 | 0 | 0 | 55000 | 220000 |
| 7 Days | 0 | 0 | 0 | 40000 | 200000 |
| 14 Days | 0 | 0 | 0 | 0 | 0 |
| 21 Days | 0 | 0 | 0 | 0 | 0 |
| 28 Days | 0 | 0 | 0 | 0 | 0 |

Table 11 shows the antimicrobial effect for the exemplary embodiment 26 in accordance with Table 8. 0.01 wt. % glass powder with a particle size of d50=4 μm of the exemplary embodiment 26 were measured in an aqueous suspension.

TABLE 11
Antibacterial effect of the powder as per Europ. Pharmakopoe
(3^{rd} edition) in 0.01 wt. % aqueous suspension:
Exemplary embodiment 26 as per Table 8; particle size 4 μm

| | E. coli | P. aeruginosa | S. aureus | C. albicans | A. niger |
|---|---|---|---|---|---|
| Start | 240000 | 340000 | 240000 | 330000 | 280000 |
| 2 Days | 0 | 100 | 100 | 32000 | 260000 |
| 7 Days | 0 | 0 | 0 | 12000 | 240000 |
| 14 Days | 0 | 0 | 0 | 4400 | 200000 |
| 21 Days | 0 | 0 | 0 | 1000 | 140000 |
| 28 Days | 0 | 0 | 0 | 1000 | 140000 |

As another especially preferred glass composition, sulfophosphate glasses can be used as additives to dental materials. These type of glasses are specified in Tables 13 through 15.

TABLE 13
Compositions (synthesis values) [wt. %] of glass compositions in accordance with the invention

| | Version 37 | Version 38 | Version 39 | Version 40 | Version 41 | Version 42 | Version 43 | Version 44 |
|---|---|---|---|---|---|---|---|---|
| P₂0₅ | 33.5 | 32.5 | 35 | 35.9 | 32.5 | 32.5 | 32.5 | 35 |
| SO₃ | 15 | 15 | 16 | 14 | 15 | 15 | 15 | 15 |
| B₂O₃ | | | | | | | | |
| Al₂O₃ | | | | | | | | |
| SiO₂ | | | | | | | | |
| Li₂O | | | | | | | | |
| Na₂O | 14.6 | 14.6 | 12.999 | 14.6 | 14.5 | 14.6 | 14.6 | 15 |
| K₂O | | | | | | | | |
| CaO | 3.3 | 3.3 | 2.4 | 35 | 11 | 3.3 | 3.3 | 10 |
| MgO | | | | | | | | |
| SrO | | | | | | | | |
| BaO | | | | | | | | |
| ZnO | 33.6 | 33.6 | 33.6 | | 26.5 | 33.6 | 33.6 | 25 |
| Ag₂O | | 1 | 0.0001 | 0.5 | 0.5 | 0.1 | | |
| CuO | | | | | | 0.3 | | |
| GeO₂ | | | | | | | | |
| TeO₂ | | | | | | | | |
| Cr₂O₃ | | | | | | 0.6 | | |
| J | | | | | | | 1 | |

TABLE 14
Antibacterial effect of the powder as per Europ. Pharmakopoe
(3^{rd} edition) in 0.001 wt. % of a glass powder in accordance
with exemplary embodiment 38 with a medium
particle size of 4 μm in aqueous suspension.

| | E. coli | P. aeruginosa | S. aureus | C. albicans | A. niger |
|---|---|---|---|---|---|
| Start | 270000 | 260000 | 260000 | 240000 | 240000 |
| 2 Days | 0 | 0 | 0 | 0 | 160000 |
| 7 Days | 0 | 0 | 0 | 0 | 160000 |
| 14 Days | 0 | 0 | 0 | 0 | 140000 |
| 21 Days | 0 | 0 | 0 | 0 | 120000 |
| 28 Days | 0 | 0 | 0 | 0 | 10000 |

Table 15 shows the antimicrobial effect of a glass powder in accordance with exemplary embodiment 38 in a 0.1 wt.-% aqueous suspension.

| | E. coli | P. aeruginosa | S. aureus | C. albicans | A. niger |
|---|---|---|---|---|---|
| Start | 250000 | 210000 | 240000 | 270000 | 280000 |
| 2 Days | 0 | 0 | 0 | 0 | 140000 |
| 7 Days | 0 | 0 | 0 | 0 | 20000 |
| 14 Days | 0 | 0 | 0 | 0 | 1500 |
| 21 Days | 0 | 0 | 0 | 0 | 100 |
| 28 Days | 0 | 0 | 0 | 0 | 100 |

Additives of dental materials can also be obtained based on silicon glasses. These types of glasses are specifed in Table 16.

TABLE 16
Compositions (synthesis values) [wt. %] of glass compositions
in accordance with the invention

| wt. % | A45 | A46 | A47 | A48 | A49 | A50 | A51 | A52 | A53 | A54 | A55 |
|---|---|---|---|---|---|---|---|---|---|---|---|
| SiO₂ | 71.00 | 45.00 | 44.50 | 35.00 | 34.90 | 44 | 60 | 59 | 47 | 45 | 46.5 |
| Na₂O | 14.10 | 22.00 | 24.50 | 27.50 | 29.50 | 24.50 | 20 | 20 | 26.5 | 24.50 | 26.5 |
| CaO | 10.00 | 22.00 | 24.50 | 27.50 | 29.50 | 24.50 | 20 | 20 | 26.5 | 24.50 | 26.5 |
| P₂0₅ | | 6.00 | 6.00 | 5.80 | 6.00 | 6.00 | | | | 6.00 | |
| Al₂O₃ | | | | | | | | | | | |
| MgO | 4.70 | | | | | | | | | | |
| Ag₂O | 0.2 | | 0.50 | 0.2 | 0.10 | 1 | | 1 | | | 0.5 |
| AgJ | | | | | | | | | | | |
| NaJ | | | | | | | | | | | |
| TiO₂ | | | | | | | | | | | |
| K₂O | | | | | | | | | | | |
| ZnO | | 5.0 | | 4.0 | | | | | | | |

Table 17 shows the ion release for Ag in mg/L under lixivation after 1 hour and after 24 hours in accordance with exemplary embodiment 12, 12c, 19, 26, 33 and 36 (see Table 8) with a particle size of 5 μm, in an aqueous suspension and a concentration of 1 wt. %.

TABLE 17

| Silver Release in mg/L | 1 hour | 24 hours |
|---|---|---|
| Version 12 | 9 | 10.8 |
| Version 12-c | 32.9 | 68.6 |
| Version 15 | 28.5 | 23.5 |
| Version 19 | 28.5 | 50.5 |
| Version 25 | 2.3 | 11 |
| Version 26 | 2.9 | 17 |
| Version 33 | 2.2 | 6.4 |
| Version 36 | 7.89 | 47.4 |

As can be seen from Table 17 in connection with Table 18, the release rate can be adjusted through the glass compositions, through the degree of ceramitation as well as through the silver concentration.

Table 18 specifies other compositions in wt. % for dental glass fillers, which can be used in glasionomers, as described in Table 19. Except for exemplary embodiment 70, all of the dental glass fillers in accordance with Table 18 have an antimicrobial effect. Table 18 also shows the thermal length expansion (CTE), the refractive index nD, the transformation temperature Tg, the radio opacity for a 2-mm-thick sample (import for dental fillers), the silver ion release (AG release) and the onset OD.

TABLE 18
Compositions for dental glass fillers

| | A56 | A57 | A58 | A59 | A60 | A61 | A62 | A63 |
|---|---|---|---|---|---|---|---|---|
| SiO₂ | 60 | 50 | 99.5 | 45 | 30 | 30 | 30 | 50 |
| Al₂°O₃ | 20 | 20 | | 10 | 30 | 20 | 20 | 9.9 |
| B₂°O₃ | | | | 10 | | | | 10 |
| ZnO | | | | | | 15 | 10 | |
| BaO | | | | 35 | | | | 30 |
| CaO | | | | | 10 | | | |
| SrO | | | | | | | | |
| P₂°O₅ | | 5 | | | 9.5 | 3 | | |
| La₂°O₃ | | 10 | | | | | | |
| ZrO₂ | 5 | 5 | | | | | | |
| Li₂O | 5 | 5 | | | | | | |
| MgO | 5 | | | | | | | |
| K₂O | 1 | | | | | | | |
| Na₂O | | | | | | 2 | | |
| ZrO₂ | | | | | | | | |
| TiO₂ | | | | | | | | |
| Nb₂°O₃ | | | | | | | | |
| Ta₂°O₅ | 1 | 1 | | | | | | |
| WO₃ | | | | | | | | |
| SrO | | | | | | 20 | 20 | |
| Ag₂O | 1 | 2 | 0.5 | 1 | 0.5 | | 2 | 0.1 |
| F | | | | 1 | 10 | 10 | 18 | |
| CTE | c.a 1 | c.a 1 | 0.6 | | 10 | 7 | 7 | 5 |
| (−30/+70) | | | | | | | | |
| 10−6/K | | | | | | | | |
| nD | 1.52 | 1.58 | 1.46 | 1.56 | 1.47 | 1.51 | 1.51 | 1.55 |
| Tg ISO | >800 | >800 | indefinable | | 440 | 512 | 505 | 630 |
| 7884-8 | | | | | | | | |
| Density | 2.6 | 2.9 | 2.2 | | 2.6 | 3.1 | 3.1 | 3 |
| (g/cm³) | | | | | | | | |
| Radio | 1.5 | 4.4 | c.a. 5 | c.a. | c.a.1 | c.a. 5 | c.a. 5 | 4.8 |
| Opacity | (75%) | (220%) | (220%) | 260 | (50%) | (250%) | (250%) | (240%) |
| (ISO 4049) | | | | | | | | |
| 2 mm glass | | | | | | | | |
| thickness) | | | | | | | | |
| AG Release | | 0.031 | | | | | 0.042 | |
| (mg/L) after | | | | | | | | |
| 24 hours | | | | | | | | |
| Onset OD | | 18.5 | | | | 16.8 | 18.2 | 5.7 |
| (absolute) | | | | | | | | |
| Assessment | | ∘ | | | | ∘ | ∘ | □ |

| | A64 | A65 | A66 | A67 | A68 | A69 | A70 |
|---|---|---|---|---|---|---|---|
| SiO₂ | 50 | 54.5 | 50 | 60 | 30 | 5 | 55 |
| Al₂°O₃ | 10 | 10 | 15 | 14 | 5 | | 10 |
| B₂°O₃ | 10 | 10 | 15 | 15 | 5 | 19.9 | 10 |
| ZnO | | | | | 10 | 20 | |
| BaO | 30 | 25 | | | | | 25 |
| CaO | | | | | 5 | | |
| SrO | | | | | | | |
| P₂°O₅ | | | | | | | |
| La₂°O₃ | | | | | 5 | 35 | |
| ZrO₂ | | | | | | | |
| Li₂O | | | | | | | |
| MgO | | | | | | | |
| K₂O | | | | | | | |
| Na₂O | | | | | 5 | | |
| ZrO₂ | | | | | 10 | | |
| TiO₂ | | | | | | 5 | |
| Nb₂°O₃ | | | | | | 10 | |
| Ta₂°O₅ | | | | | | | |
| WO₃ | | | | | | 5 | |
| SrO | | | 20 | 15 | 25 | | |
| Ag₂O | 1 | 0.5 | 1 | 1 | | 0.1 | |
| F | 1 | | | 2 | | | |
| CTE | 5 | 4 | 4 | 3 | 8 | 6 | 4 |
| (−30/+70) | | | | | | | |
| 10−6/K | | | | | | | |
| nD | 1.53 | 1.53 | 1.52 | 1.5 | 1.6 | 1.83 | 1.53 |
| Tg ISO | 595 | 630 | 680 | 610 | 530 | 585 | 630 |
| 7884-8 | | | | | | | |
| Density | 2.9 | 2.8 | 2.6 | 2.46 | 3.42 | 4.55 | 2.8 |
| (g/cm³) | | | | | | | |
| Radio | 4.8 | 4.2 | 4.2 | c.a. 4 | c.a. 6 | c.a. 8 | 4.2 |
| Opacity | (240%) | (210%) | (210%) | (200%) | (300%) | (400%) | (210%) |
| (ISO 4049) | | | | | | | |
| 2 mm glass | | | | | | | |
| thickness) | | | | | | | |
| AG Release | | | 0.039 | | | | |
| (mg/L) after | | | | | | | |
| 24 hours | | | | | | | |
| Onset OD | | 6.8 | 15.9 | | | | 2.8 |
| (absolute) | | | | | | | |
| Assessment | | ▾ | ∘ | | | | ⋄ |

▾ slightly antibacterial
□ very slightly antibacterial
∘ antibacterial
⋄ no activity

Below are examples for compositions in accordance with the invention for glasionomer cements.

The information refers to the wt. % of the total composition.

| Glasionomer with antimicrobial glass powder | |
|---|---|
| or | |
| Glasionomer with antimicrobial effect | Aqueous components |
| 50 wt. % | 50 wt. % polyacrylic acid |
| 47.5 wt. % | 47.5 wt. % polyacrylic acid |
| | 5 wt. % tartaric acid |
| 45 wt. % | 45 wt. % polyacrylic acid |
| | 5 wt. % tartaric acid |
| | 5 wt. % CH₃OH |
| 75 wt. % | 15 wt. % polyacrylic acid |
| | 10 wt. % tartaric acid |
| 64.3 wt. % | 25.7 wt. % polyacrylic acid |
| | 10 wt. % tartaric acid |

All glass powders named here with antimicrobial effects can be used in the aforementioned compositions. Even mixtures of antimicrobial glass powders with conventional glass powders are possible. The share of antimicrobial glass powder in the mixture with conventional glasionomers is preferably 0.5 to 25 wt. %, more preferably 5 to 15 wt. %. Alternatively, the glasionomer itself can be an antimicrobial glass powder.

The following table 19 specifies exemplary embodiments, in which a methacrylate monomer (a so-called Bis-GMA) with a non-antimicrobial dental glass filler A70 in accordance with Table 18 and an antimicrobial dental glass filler in the specified concentration in accordance with Tables 1, 2, 8, 13 and 18 were combined into a glasionomer cement.

TABLE 19
Components for a glasionomer cement in wt. % of the total composition

| Monomer Sample | | | | | | Onset OD | | Ag Release |
|---|---|---|---|---|---|---|---|---|
| Bis GMA | A70 | AM-Powder | | Transparency | Translucence | (absolute | | After 24 |
| [%] | [%] | Glass | [%] | [%] | [%] | values) | Assessment | Hours |
| 100 | | | | 92.1 | 77.9 | 1.9 | ⋄ | |
| 50 | 50 | | | 52.2 | 26.6 | 1.8 | ⋄ | |
| 50 | 45 | A46 | 5 | 51.4 | 26.5 | 5.9 | ▾ | |
| 50 | 48 | A46 | 2 | 51.6 | 26.3 | 2.9 | ● | |
| 50 | 20 | A21 | 30 | 51.5 | 28.8 | 15.3 | ∘ | |
| 50 | 35 | A21 | 15 | 51.4 | 27.3 | 6.2 | ▾ | |
| 50 | 45 | A26 | 5 | 51.0 | 27.8 | | ∘ | 0.029 |
| 50 | 48 | A26 | 2 | 51.7 | 27.4 | | ∘ | 0.018 |
| 50 | 45 | A16 | 5 | 39.9 | 18.5 | 18.9 | ∘ | 0.046 |
| 50 | 48 | A16 | 2 | 45.9 | 23.1 | 16.1 | ∘ | 0.035 |
| 50 | 45 | A12-c | 5 | 33.2 | 16.5 | 17.7 | ∘ | 0.041 |
| 50 | 48 | A12-c | 2 | 42.9 | 22.7 | 15.9 | ∘ | 0.029 |
| 50 | 45 | A27 | 5 | 50.1 | 26.7 | 15.6 | ∘ | |
| 50 | 48 | A27 | 2 | 49.1 | 25.0 | 14.9 | ∘ | |
| 50 | 45 | A33 | 5 | 49.9 | 26.7 | 15.3 | ∘ | |
| 50 | 48 | A33 | 2 | 51.4 | 27.0 | 6.2 | ▾ | |
| 50 | 45 | A17 | 5 | 40.2 | 17.4 | | | |
| 50 | 48 | A17 | 2 | 45.3 | 21.7 | | | |

▾ slightly antibacterial
□ very slightly antibacterial
∘ antibacterial
● very slight activity
⋄ no activity

Table 20 shows the observed proliferation over 48 hours for a glass powder with a particle size between d50 from 4 μm and a glass composition in accordance with 1, which was introduced homogenously in the specified concentration (wt. %) to the cement.

Onset OD is the optical density in the surrounding nutrient solution. The transmission of the nutrient solution is disturbed through proliferation (formation of daughter cells) and the release of the cells form the surface into the surrounding nutrient solution. This absorption at certain wavelengths correlates with the antimicrobial effectiveness of the surface. The higher the onset OD value, the stronger the antimicrobial effectiveness of the surface.

1. Use of glass compositions with antimicrobial and/or disinfectant effect in materials for tooth restoration, excepting implants, in the field of fillings, wherein the filling is a material selected from the following group:
a composite material
a glasionomer cement
a compomer,
wherein the glass composition comprises the following components (in percentage by weight on an oxide basis)

| | |
|---|---|
| SiO₂ | 0-99.5 percent by weight |
| P₂O₅ | 0-80 percent by weight |
| SO₃ | 0-40 percent by weight |
| B₂0₃ | 0-80 percent by weight |
| Al₂O₃ | 0-30 percent by weight |
| Li₂O | 0-30 percent by weight |
| Na₂O | 0-40 percent by weight |
| K₂O | 0-30 percent by weight |
| CaO | 0-25 percent by weight |
| MgO | 0-15 percent by weight |
| SrO | 0-30 percent by weight |
| BaO | 0-40 percent by weight |
| ZnO | 0-<15 percent by weight |
| TiO₂ | 0-10 percent by weight |
| ZrO₂ | 0-15 percent by weight |
| CeO₂ | 0-10 percent by weight |
| Ag₂O | 0.01-5 percent by weight |
| F | 0-70 percent by weight |
| J | 0-10 percent by weight |
| Fe₂O₃ | 0-5 percent by weight |

and if necessary trace elements and/or normal refining agents in commodity quantities, wherein the sum of SiO₂+P₂O₅+SO₃+B₂O₃+Al₂O₃ is greater than 20 percent by weight and a maximum of 99.5 percent by weight and the sum of ZnO+Ag₂O+CuO+GeO₂+TeO₂+Cr₂O₃>0.01 percent by weight.

2. Use of glass compositions with antimicrobial and/or disinfectant effect in materials for tooth restoration, excepting implants, wherein the glass composition comprises the following components (in percentage by weight on an oxide basis):

| | |
|---|---|
| SiO₂ | 0-99.5 percent by weight, preferably |
| | 0-80 percent by weight |
| P₂O₅ | 0-80 percent by weight |
| SO₃ | 0-40 percent by weight |
| B₂0₃ | 0-80 percent by weight |
| Al₂O₃ | 0-30 percent by weight |
| Li₂O | 0-30 percent by weight |
| Na₂O | 0-40 percent by weight |
| K₂O | 0-30 percent by weight |
| CaO | 0-25 percent by weight |
| MgO | 0-15 percent by weight |
| SrO | 0-30 percent by weight |
| BaO | 0-40 percent by weight |
| ZnO | 0-<15 percent by weight, preferably |
| | 5-<15 percent by weight |
| F | 0-65 percent by weight |
| J | 0-10 percent by weight |
| Fe₂O₃ | 0-5 percent by weight |
| Ag₂O | 0.01-5 percent by weight |

and if necessary trace elements and/or normal refining agents in commodity quantities, wherein the sum of SiO₂+P₂O₅+SO₃+B₂O₃+Al₂O₃ is greater than 20 percent by weight and a maximum of 99.5 percent by weight, in particular a maximum of 80 percent by weight.

3. Application according to claim 1 in coating, filling or veneering materials for ceramic dental superstructures.

4. Application according to claim 1, characterized in that the glass composition comprises ZnO in the range of 0.25 to <15 percent by weight, preferably 2.5 to 10 percent by weight.

5. Application according to claim 1, characterized in that the glass composition comprises Ag₂O in the range of 0.05 to 2 percent by weight, in particular preferably 0.5 to 2 percent by weight.

6. Application according to claim 1, characterized in that the sum BaO+SrO is greater than 10 percent by weight.

7. Ion-releasing glass composition with antimicrobial effect for application as materials for tooth restoration, in particular in materials for fillings, in combination with materials for fillings, in particular selected from glasionomers, composites, compomers, wherein the glass composition comprises the following components (in percent by weight on an oxide basis):

| | |
|---|---|
| P₂O₅ | >66-80 percent by weight |
| SO₃ | 0-40 percent by weight |
| B₂0₃ | 0-1 percent by weight |
| Al₂O₃ | >6.2-10 percent by weight |
| SiO₂ | 0-10 percent by weight |
| Li₂O | 0-25 percent by weight |
| Na₂O | 9-20 percent by weight |
| CaO | 0-25 percent by weight |
| MgO | 0-15 percent by weight |
| SrO | 0-15 percent by weight |
| BaO | 0-15 percent by weight |
| ZnO | 0-<15 percent by weight |
| Ag₂O | 0-5 percent by weight |
| CuO | 0-10 percent by weight |
| GeO₂ | 0-10 percent by weight |
| TeO₂ | 0-15 percent by weight |
| Cr₂O₃ | 0-10 percent by weight |
| J | 0-10 percent by weight |
| F | 0-3 percent by weight |

wherein the sum of ZnO+Ag₂O+CuO+GeO₂+TeO₂+Cr₂O₃+J>0.01 percent by weight.

8. Ion-releasing glass composition with antimicrobial effect for application as materials for tooth restoration, in particular in materials for fillings, in combination with materials for fillings, in particular selected from glasionomers, composites, compomers, wherein the glass composition comprises the following components (in percent by weight on an oxide basis):

| | |
|---|---|
| P₂O₅ | >66-80 percent by weight |
| SO₃ | 0-40 percent by weight |
| B₂0₃ | 0-1 percent by weight |
| Al₂O₃ | 0-3.9 percent by weight |
| SiO₂ | 0-10 percent by weight |
| CaO | 0-25 percent by weight |
| MgO | 0-15 percent by weight |
| SrO | 0-15 percent by weight |
| BaO | 0-15 percent by weight |
| ZnO | 0-<15 percent by weight |
| Ag₂O | 0-5 percent by weight |
| CuO | 0-10 percent by weight |
| GeO₂ | 0-10 percent by weight |
| TeO₂ | 0-15 percent by weight |
| Cr₂O₃ | 0-10 percent by weight |
| J | 0-10 percent by weight |
| F | 0-3 percent by weight |

wherein the sum of ZnO+Ag₂O+CuO+GeO₂+TeO₂+Cr₂O₃+J>1 percent by weight.

9. Ion-releasing glass composition with antimicrobial effect for application as materials for tooth restoration, in particular in materials for fillings, in combination with materials for fillings, in particular selected from glasionomers, composites, compomers, wherein the glass composition comprises the following components (in percent by weight on an oxide basis):

| | |
|---|---|
| P₂O₅ | >45-90 percent by weight |
| B₂0₃ | 0-60 percent by weight |
| SiO₂ | 0-40 percent by weight |
| Al₂O₃ | 0-20 percent by weight |
| SO₃ | 0-30 percent by weight |
| Li₂O | 0-0.1 percent by weight |
| Na₂O | 0-0.1 percent by weight |
| K₂O | 0-0.1 percent by weight |
| CaO | 0-40 percent by weight |
| MgO | 0-40 percent by weight |
| SrO | 0-15 percent by weight |
| BaO | 0-40 percent by weight |
| ZnO | 0-<15 percent by weight |
| Ag₂O | 0-5 percent by weight |
| CuO | 0-15 percent by weight |
| Cr₂O₃ | 0-10 percent by weight |
| J | 0-10 percent by weight |
| TeO₂ | 0-10 percent by weight |
| GeO₂ | 0-10 percent by weight |
| TiO₂ | 0-10 percent by weight |
| ZrO₂ | 0-10 percent by weight |
| La₂O₃ | 0-10 percent by weight |
| Nb₂O₃ | 0-5 percent by weight |
| CeO₂ | 0-5 percent by weight |
| Fe₂O₃ | 0-5 percent by weight |
| WO₃ | 0-5 percent by weight |
| Bi₂O₃ | 0-5 percent by weight |
| MoO₃ | 0-5 percent by weight |

wherein the sum of ZnO+Ag₂O+CuO+GeO₂+TeO₂+Cr₂O₃+J>0.001 percent by weight.

10. Ion-releasing glass composition with antimicrobial effect for application as materials for tooth restoration, in particular in materials for fillings, in combination with materials for fillings, in particular selected from glasionomers, composites, compomers, wherein the glass composition comprises the following components (in percent by weight on an oxide basis):

| | |
|---|---|
| SiO₂ | 40-80 percent by weight |
| B₂0₃ | 5-40 percent by weight |
| Al₂O₃ | 0-10 percent by weight |
| P₂O₅ | 0-30 percent by weight |
| Li₂O | 0-25 percent by weight |
| Na₂O | 0-25 percent by weight |
| K₂O | 0-25 percent by weight |
| CaO | 0-25 percent by weight |
| MgO | 0-15 percent by weight |
| SrO | 0-15 percent by weight |
| BaO | 0-15 percent by weight |
| ZnO | 0-<15 percent by weight |
| Ag₂O | 0.01-5 percent by weight |
| CuO | 0-10 percent by weight |
| GeO₂ | 0-10 percent by weight |
| TeO₂ | 0-15 percent by weight |
| Cr₂O₃ | 0-10 percent by weight |
| J | 0-10 percent by weight |
| F | 0-10 percent by weight |

wherein the sum of ZnO+Ag₂O+CuO+GeO₂+TeO₂+Cr₂O₃+J ranges between 5 and 70 percent by weight.

11. Glass composition according to claim 7, characterized in that the glass composition comprises ZnO in the range of 0.25 to <15 percent by weight, preferably 2.5 to 10 percent by weight.

12. Glass composition according to claim 7, characterized in that the glass composition comprises Ag₂O in the range of 0.05 to 2 percent by weight, preferably 0.5 to 2 percent by weight.

13. Glass composition according to claim 7, characterized in that the glass composition contains BaO and SrO and the sum of BaO+SrO is greater than 10 percent by weight.

14. Ion-releasing glass composition according to claim 7, characterized in that at least two vitreous phases are formed in the glass composition.

15. Ion-releasing glass composition according to claim 14, characterized in that in the glass composition at least two vitreous phases exhibit different compositions.

16. Ion-releasing glass composition according to claim 14, characterized in that the glass composition is a borosilicate glass composition.

17. Ion-releasing glass ceramic with antimicrobial effect for application as materials for tooth restoration, in particular in materials for fillings, in combination with materials for fillings, in particular selected from glasionomers, composites, compomers, wherein the base glass of the glass ceramic comprises the following components (in percent by weight on an oxide basis):

| | |
|---|---|
| SiO₂ | 20-90 percent by weight |
| CaO | 0-45 percent by weight |
| Na₂O | 0-40 percent by weight |
| P₂O₅ | 0-15 percent by weight |
| Ag₂O | 0.01-5 percent by weight |
| ZnO | 0-20 percent by weight |

wherein the sum of ZnO+Ag₂O+CuO+GeO₂+TeO₂+Cr₂O₃+J is greater than 0.001 percent by weight.

18. Ion-releasing glass ceramic according to claim 17, characterized in that the crystalline main phases comprise alkali-alkaline earth-silicate and/or alkali-silicate and/or alkaline earth-silicate, excepting a glass ceramic with the sole crystalline main phase 1 Na2O.2 CaO.3 SiO2 and the main phase Na₄Ca₃Si₈O₁₆ (OH₂).

19. Method for the production of an ion-releasing glass composition according to claim 14, characterized in that the at least two phases are preserved by means of tempering in a temperature range Tg≦T≦Tg+300° C., wherein Tg is the transformation temperature of the glass.

20. Method for the production of an ion-releasing glass composition according to claim 17, characterized in that the base glass for the glass ceramic is ground and subsequent to that a ceramizing of the powdery base glass takes place.

21. Method for the production of an ion-releasing glass composition according to claim 17, characterized in that the base glass for the glass ceramic is ceramized first and is ground subsequent to that.

22. Glasionomer cement for dental applications comprising:
a polymer which contains free carboxylic acid groups,
an ion-releasing glasionomer glass composition as well as an ion-releasing antimicrobial glass composition or
an ion-releasing antimicrobial glass ceramic according to claim 7.

23. Glasionomer cement according to claim 22 characterized in that 1-90 percent by weight of the total composition is an ion-releasing glass/glass ceramic composition, wherein the ion-releasing glass composition comprises an ion-releasing antimicrobial glass or an ion-releasing glass ceramic or is a mixture of ion-releasing glasionomer composition with an ion-releasing antimicrobial glass composition or an ion-releasing glass ceramic.

24. Glasionomer cement according to claim 22, characterized in that the Ag₂O content >0.01 percent by weight.

25. Glassionomer cement according to claim 22, characterized in that the ratio of antimicrobial glass composition/glasionomer cement and/or fillings >0.001.

26. Glassionomer cement according to claim 22, characterized in that the ratio of antimicrobial glass composition/glasionomer cement and/or fillings <200, preferably less than 100, quite preferably less than 10.

27. Coating or veneering material for ceramic dental superstructures, comprising
a base material, preferably a filling, in particular selected from:
a composite material
a glasionomer cement,
a compomer,
an ion-releasing antimicrobial glass composition or an ion-releasing glass ceramic according to claim 7.
